(19) United States
(12) Patent Application Publication
(10) Pub. No.: US 2006/0292614 A1
(43) Pub. Date: Dec. 28, 2006

(54) METHODS OF GENE EXPRESSION MONITORING

(75) Inventors: Yanxiang Cao, Mountain View, CA (US); Lubert Stryer, Stanford, CA (US); David Lockhart, Del Mar, CA (US); Catherine G. Dulac, Cambridge, MA (US); Ian Tietjen, Somerville, MA (US); Jason Rihel, Somerville, MA (US)

Correspondence Address: BANNER & WITCOFF LTD.,;COUNSEL FOR AFFYMETRIX, 1001 G STREET , N.W., ELEVENTH FLOOR, WASHINGTON, DC, 20001-4597, US

(73) Assignee: Affymetrix, Inc., Santa Clara, CA (US)

(21) Appl. No.: 11/467,971

(22) Filed: Aug. 29, 2006

Related U.S. Application Data
(63) Continuation of application No. 09/634,352, filed on Aug. 9, 2000.
(60) Provisional application No. 60/160,925, filed on Oct. 22, 1999.
(60) Provisional application No. 60/148,081, filed on Aug. 9, 1999.

(51) Int. Cl. C12Q 1/68

(52) U.S. Cl. 435/6

(57) ABSTRACT

The invention provides methods of monitoring expression of a plurality of genes in a cell or small population of cells. Preferred methods entail contacting an array of probes with a population of nucleic acids derived from a population of fewer than 1000 cells then determining the relative hybridization of the probes to the population of nucleic acid as a measure of the relative representation of genes from the cells. The invention further provides methods of classifying cells. These preferred methods entail determining an expression profile of each of a plurality of cells then classifying the cells in clusters determined by similarity of expression profile. The invention further provides methods of monitoring differentiation of a cell lineage. These preferred methods entail determining an expression profile of each of a plurality of cells at different differentiation stages within the lineage. These cells can then be classified into clusters determined by similarity of expression profile. The clusters can then be ordered by similarity of expression profile. A time course of expression levels for each of the plurality of genes at different stages of differentiation in the cell lineage can then be determined.

This application claims the benefit of the filing dates of U.S. patent application Ser. No. 09/634,352, filed Aug. 9, 2000; U.S. provisional application Ser. No. 60/148,081 filed Aug. 9, 1999; and U.S. provisional application Ser. No. 60/160,925 filed Oct. 22, 1999, each of which is hereby incorporated by reference in its entirety for all purposes.

### BACKGROUND OF THE INVENTION

The identification of genes associated with development, differentiation, disease states, and response to cellular environment is an important step for advanced understanding of these phenomena. Specifically, effective methods for conducting genetic analysis are needed to identify and isolate genes that are differentially expressed in various cells or under altered cell environments and to further elucidate functional genetic networks.

Many disease states are characterized by differences in the expression levels of various genes either through changes in the copy number of the genetic DNA or through changes in levels of transcription (e.g., through control of initiation, provision of RNA precursors, or RNA processing) of particular genes. For example, losses and gains of genetic material play an important role in malignant transformation and progression. These gains and losses are thought to be caused by at least two kinds of genes. Oncogenes are positive regulators of tumorgenesis, while tumor suppressor genes are negative regulators of tumorgenesis (Marshall, Cell 64:313-326 (1991); Weinberg, Science 254: 1138-1146 (1991). Therefore, one mechanism of activating unregulated growth is to increase the number of genes coding for oncogene proteins or to increase the level of expression of these oncogenes (e.g., in response to cellular or environmental changes), and another is to lose genetic material or to decrease the level of expression of genes that code for tumor suppressors. This model is supported by the losses and gains of genetic material associate with glioma progression (Michelson et al., J. Cellular Biochrome. 46: 3-8 (1991)). Thus, changes in the expression (transcription) levels of particular genes (e.g. oncogenes or tumor suppressors), serve as signposts for the presence and progression of various cancers.

Similarly, control of the cell cycle and cell development, as well as diseases, are characterized by the variations in the transcription levels of particular genes. Thus, for example, a viral infection is often characterized by the elevated expression of genes of the particular virus. For example, outbreaks of Herpes simplex, Epstein-Barr virus infections (e.g., infectious mononucleosis), cytomegalovirus, Varicella-zoster virus infections, parvovirus infections, human papillomavirus infections, are all characterized by elevated expression of various genes present in the respective virus. Detection of elevated expression levels of characteristic viral genes provides an effective diagnostic of the disease state. In particular, viruses such as herpes simplex, enter quiescent states for periods of time only to erupt in brief periods of rapid replication. Detection of expression levels of characteristic viral genes allows detection of such active proliferative (and presumably infective) states.

In addition, expression of characteristic genes by cell subpopulation within a normal or abnormal tissue is indicative of cell potentials that are therapeutically important. For example, identification of genes encoding specific surface molecules, growth factor receptors or nuclear proteins have led to the characterization of rare cell populations with stem cell potentials in the adult bone marrow, muscle and brain.

The development of VLSIPS™ technology provided methods for synthesizing arrays of many different probes that can occupy a very small surface area. See U.S. Pat. No. 5,143,854 and WO 90/15070. WO 92/10588 describes methods for making arrays of probes that can be used for sequence analysis of a target nucleic acid and to detect the presence of a nucleic acid containing a specific nucleotide sequence.

### SUMMARY OF THE INVENTION

The invention provides methods of monitoring expression of a plurality of genes in a cell, one or more cells or a small population of cells. Preferred methods entail contacting an array of probes with a population of nucleic acids derived from a population of fewer than 1000 cells then determining the relative hybridization of the probes to the population of nucleic acid as a measure of the relative abundance of specific mRNAs in the cells.

The invention further provides methods of classifying cells. These preferred methods entail determining an expression profile of each of a plurality of cells then classifying the cells in clusters determined by similarity of expression profile.

The invention further provides methods of monitoring differentiation of a cell lineage. These preferred methods entail determining an expression profile of each of a plurality of cells at different differentiation stages within the lineage. These cells can then be classified into clusters determined by similarity of expression profile. The clusters can then be ordered by similarity of expression profile. A time course of expression levels for each of the plurality of genes at different stages of differentiation in the cell lineage can then be determined.

The invention further provides methods to identify the nature and function of cells. These preferred methods entail comparing the gene expression profiles of each of a plurality of cells in order to determine the nature and function of the cells.

Embodiments of the present invention are further directed to methods of diagnosing cell samples such as normal, malignant, cancerous or precancerous cells by comparing the gene expression profiles of cells to the known gene expression profiles of normal, malignant, cancerous or precancerous cells. Embodiments of the present invention further include a method of identifying a specific cell type by determining an expression profile of a plurality of cells, classifying the cells in clusters determined by similarity of expression profile and then determining the nature and function of a plurality of cells. The cells can originate from any tissue source including that from the adult brain and peripheral sensory organs. In addition, the cells can be deduced to have stem cell potentials. The cells may be obtained from a biopsy without in vitro propagation of the cells. The cells may further be obtained from a tissue known or suspected to be neoplastic.

### BRIEF DESCRIPTION OF THE DRAWINGS

In the course of the detailed description of certain preferred embodiments to follow, reference will be made to the attached drawings, in which,

FIG. 1 is a comparison of GENECHIP expression arrays showing gene expression profiling results in main olfactory epithelium versus gene expression in a single olfactory sensory neuron.

FIG. 2 is an enlargement of a region of the GENECHIP expression arrays of FIG. 1.

FIG. 3 shows GENECHIP expression array patterns of signature molecules expressed in the retina.

FIG. 4 shows GENECHIP expression array patterns of signature or representative molecules expressed in a single photoreceptor cell of the retina.

FIG. 5 is a chart showing correlation coefficients of expression profiles between newborn neuron cells at different development stages.

FIG. 6 is a schematic representing clustering of cells by similarity of expression profiles.

FIG. 7 is a graph of the percent of genes expressed in olfactory epithelium and single olfactory neurons versus the expression level

FIG. 8 is a chart of the correlation of gene expression profiles by Southern Blot and microarray hybridization.

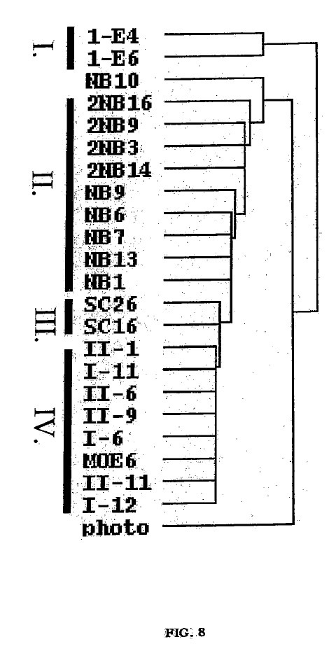

FIG. 9 shoes the gene cluster identifying an embryonic cell as a supporting cell and the corresponding gene expression in the tissue.

FIG. 10 shows specific gene expression profiles of individual neurons that cannot be detected by monitoring gene expression in the whole tissue.

### DEFINITIONS

The terms “nucleic acid” or “nucleic acid molecule” refer to a deoxyribonucleotide or ribonucleotide polymer in either single- or double-stranded form, and unless otherwise limited, can encompass known analogs of natural nucleotides that can function in a similar manner as naturally occurring nucleotides.

A polynucleotide probe is a single stranded nucleic acid capable of binding to a target nucleic acid of complementary sequence through one or more types of chemical bonds, usually through complementary base pairing, usually through hydrogen bond formation. A polynucleotide probe can include natural (i.e., A, G, C, or T) or modified bases (e.g., 7-deazaguanosine, inosine). Therefore, polynucleotide probes can be between about 5-10,000, 10-5,000, 10-500, 10-50, 10-25, 10-20, 15-25, and 15-20 bases long. Probes are typically about 10-50 bases long, and are often 15-25 bases. In its simplest embodiment, the array includes test probes (also referred to as polynucleotide probes) more than 5 bases long, preferably more than 10 bases long, and some more than 40 bases long. The probes can also be less than 50 bases long. In some cases, these polynucleotide probes can range from about 5 to about 45 or 5 to about 50 nucleotides long, or from about 10 to about 40 nucleotides long, or from about 15 to about 40 nucleotides in length. The probes can also be about 20 or 25 nucleotides in length.

In addition, the bases in a polynucleotide probe can be joined by a linkage other than a phosphodiester bond, so long as it does not interfere with hybridization. Thus, polynucleotide probes can be peptide nucleic acids in which the constituent bases are joined by peptide bonds rather than phosphodiester linkages. The length of probes used as components of pools for hybridization to distal segments of a target sequence often increases as the spacing of the segments increase thereby allowing hybridization to be conducted under greater stringency to increase discrimination between matched and mismatched pools of probes.

Relatively short polynucleotide probes can be sufficient to specifically hybridize to and distinguish target sequences. Therefore, the polynucleotide probes can be less than 50 nucleotides in length, generally less than 46 nucleotides, more generally less than 41 nucleotides, most generally less than 36 nucleotides, preferably less than 31 nucleotides, more preferably less than 26 nucleotides, and even more preferably less than 21 nucleotides in length. A typical probe length within the teachings of the present invention is one having 25 nucleotides. The probes can also be less than 16 nucleotides, less than 13 nucleotides in length, less than 9 nucleotides in length and less than 7 nucleotides in length.

Typically, arrays can have polynucleotides as short as 10 nucleotides or 15 nucleotides. In addition, 20 or 25 nucleotides can be used to specifically detect and quantify nucleic acid expression levels. Where ligation discrimination methods are used, the polynucleotide arrays can contain shorter polynucleotides. Arrays containing longer polynucleotides are also suitable. High density arrays can comprise greater than about 100, 1000, 16,000, 65,000, 250,000 or even greater than about 1,000,000 different polynucleotide probes.

The term “target nucleic acid” refers to a nucleic acid (often derived from a biological sample), to which the polynucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The term target nucleic acid can refer to the specific subsequence of a larger nucleic acid to which the probe is directed or to the overall sequence (e.g., gene or mRNA) whose expression level it is desired to detect. The difference in usage can be apparent from context.

“Subsequence” refers to a sequence of nucleic acids that comprise a part of a longer sequence of nucleic acids.

“Gene” refers to a unit of inheritable genetic material found in a chromosome, such as in a human chromosome. Each gene is composed of a linear chain of deoxyribonucleotides which can be referred to by the sequence of nucleotides forming the chain. Thus, “sequence” is used to indicate both the ordered listing of the nucleotides which form the chain, and the chain which has that sequence of nucleotides. The term “sequence” is used in the same way in referring to RNA chains, linear chains made of ribonucleotides. The gene includes regulatory and control sequences, sequences which can be transcribed into an RNA molecule, and can contain sequences with unknown function. Some of the RNA products (products of transcription from DNA) are messenger RNAs (mRNAs) which initially include ribonucleotide sequences (or sequence) which are translated into a polypeptide and ribonucleotide sequences which are not translated. The sequences which are not translated include control sequences, introns and sequences with unknowns function. It can be recognized that small differences in nucleotide sequence for the same gene can exist between different persons, or between normal cells and cancerous cells, without altering the identity of the gene.

“Gene expression pattern” means the set of genes of a specific tissue or cell type that are transcribed or “expressed” to form RNA molecules. Which genes are expressed in a specific cell line or tissue can depend on factors such as tissue or cell type, stage of development or the cell, tissue, or target organism and whether the cells are normal or transformed cells, such as cancerous cells. For example, a gene can be expressed at the embryonic or fetal stage in the development of a specific target organism and then become non-expressed as the target organism matures. Alternatively, a gene can be expressed in liver tissue but not in brain tissue of an adult human.

Specific hybridization refers to the binding, duplexing, or hybridizing of a molecule only to a particular nucleotide sequence under stringent conditions when that sequence is present in a complex mixture (e.g., total cellular) DNA or RNA. Stringent conditions are conditions under which a probe can hybridize to its target subsequence, but to no other sequences. Stringent conditions are sequence-dependent and are different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium. (As the target sequences are generally present in excess, at Tₘ, 50% of the probes are occupied at equilibrium). Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide or tetraalkyl ammonium salts. For example, conditions of 5× SSPE (750 mM NaCl, 50 mM Na Phosphate, 5 mM EDTA, pH 7.4) and a temperature of 25-30° C. are suitable for allele-specific probe hybridizations. (See Sambrook et al, Molecular Cloning 1989)

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe,” a “normalization control” probe, an expression level control probe and the like. A perfect match control or perfect match probe is, however, distinguished from a “mismatch control” or “mismatch probe.”

The term “mismatch control” or “mismatch probe” refer to probes whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch can comprise one or more bases. While the mismatch(s) can be located anywhere in the mismatch probe, terminal mismatches are less desirable as terminal mismatch is less likely to prevent hybridization of the target sequence.

The term “probe set” comprises at least a plurality of genes perfectly matched with a known target sequence.

The terms “background” or “background signal intensity” refer to hybridization signals resulting from non-specific binding, or other interactions, between the labeled target nucleic acids and components of the polynucleotide array (e.g., the polynucleotide probes, control probes, or the array substrate). Background signals can also be produced by intrinsic fluorescence of the array components themselves. A single background signal can be calculated for the entire array, or a different background signal can be calculated for each region of the array. In some embodiments, background is calculated as the average hybridization signal intensity for the lowest 1% to 10% of the probes in the array, or region of the array. In expression monitoring arrays (i.e., where probes are preselected to hybridize to specific nucleic acids (genes), a different background signal can be calculated for each target nucleic acid. Where a different background signal is calculated for each target gene, the background signal is calculated for the lowest 1% to 10% of the probes for each gene. Where the probes to a particular gene hybridize well and thus appear to be specifically binding to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample such as bacterial genes where the sample is of mammalian origin). Background can also be calculated as the average signal intensity produced by regions of the array that lack any probes at all.

The term “quantifying” when used in the context of quantifying nucleic acid abundance or concentrations (e.g., transcription levels of a gene) can refer to absolute or to relative quantification. Absolute quantification can be accomplished by inclusion of known concentration(s) of one or more target nucleic acids (e.g., control nucleic acids such as BioB or with known amounts the target nucleic acids themselves) and referencing the hybridization intensity of unknowns with the known target nucleic acids (e.g., through generation of a standard curve). Alternatively, relative quantification can be accomplished by comparison of hybridization signals between two or more genes, or between two or more treatments to quantify the changes in hybridization intensity and, by implication, transcription level.

The term “cluster” or “clustering” refers to clustering algorithms, such as principal components analysis and variable clustering analysis. These algorithms serve to “cluster” cells into groups. The purpose of clustering is to place the isolates into groups or clusters suggested by the data, not defined a priori, such that isolates in a given cluster tend to be similar and isolates in different clusters tend to be dissimilar. Methods of clustering are described in Tamayo et al., Proc. Natl. Acad. Sci U.S.A. (1999) 96: 2907-2912 and Eisen et al., Proc. Natl. Acad. Sci U.S.A. (1998) 95: 14863-14868 each hereby incorporated by reference in its entirety for all purposes. Software useful for clustering includes GeneCluster 1.0 provided by the Whitehead/MIT Center for Genome Research.

A small population of cells means a population of 1000 or fewer cells, typically 100 or fewer, or ten or fewer. Expression monitoring according to the present invention can be performed on a single cell.

### DETAILED DESCRIPTION OF CERTAIN PREFERRED EMBODIMENTS

The principles of the present invention may be advantageously applied to carry out methods for monitoring the expression of genes from a single cell, one or more cells or a small population of cells by contacting an array of probes with nucleic acids derived from a single cell or a population of about 1000 cells or fewer cells, and determining the relative hybridization of the probes to the nucleic acids so as to measure the relative expression of genes from the cell(s). According to one embodiment of the present invention, the array of probes includes microarrays such as the GENECHIP. According to alternate embodiments of the present invention, the array of probes includes substrates such as filters, nitrocellulose, nylon substrates and other array substrates known to those skilled in the art.

Embodiments of the present invention are also directed to methods for monitoring differential expression by contacting an array of probes with a first and a second population of nucleic acids respectively derived from a first single cell and a second single cell, and determining the relative hybridization of the probes to the nucleic acids from the first cell and the second cell to identify at least one probe hybridizing to a gene that is differentially expressed between the first cell and the second cell. According to one aspect of the method the first and second populations of nucleic acids are differentially labeled and simultaneously applied to the array of probes. Alternatively, the first and second populations of nucleic acids are applied separately to the array of probes. The array of probes includes a plurality of probes perfectly complementary to or perfectly matched to each of a plurality of known transcripts. In an alternative enbodiment, the probe may bind to a differentially expressed gene to clone the gene. According to a further embodiment, a database of nucleic acid sequences can be searched for a nucleic acid sequence that includes a sequence from a probe that hybridizes to a differentially expressed gene. The first and second cells can be at different stages of development within a common cell lineage.

Embodiments of the present invention are further directed to methods for classifying cells according to their similarity of gene expression by determining an expression profile of each of a plurality of cells by contacting an array or arrays of probes with nucleic acids derived from each cell, determining the relative hybridization of the probes to the nucleic acids so as to measure the relative expression of genes from the cells, and classifying the cells in clusters according to similarity of expression profile. Embodiments of the present invention are still further directed to methods of monitoring differentiation of a cell lineage which includes the steps of determining an expression profile of each of a plurality of cells at different stages of differentiation within the lineage by contacting an array or arrays of probes with nucleic acids derived from each cell, and determining the relative hybridization of the probes to the nucleic acids so as to measure the relative expression of genes from the cells. The cells are then classified in clusters according to similarity of expression profile, and the clusters can then be ordered by similarity of expression profile. A time course of expression levels can then be determined for each of the plurality of genes at different stages of differentiation in the cell lineage.

The methods of the present invention advantageously allow one to determine genes differentially expressed between a given first cell and a given second cell. The two cells may be at different stages of development within a common cell lineage. The methods of the present invention would allow one to compare the expression pattern and to determine what genes are expressed at different stages of development. According to the teachings of the present invention, the above described methods include the following general aspects: preparation of a sample of nucleic acids, hybridization of the sample of nucleic acids to an array, detecting the hybridized nucleic acids and, in some further aspects of the methods, analyzing the hybridization patterns.

The methods of the present invention advantageously allow one to identify the nature, the function or the state of disease of a cell by characterizing and comparing the expression profiles from a plurality of cells and alternatively, comparing expression profiles obtained according to the present invention with a database of known expression profiles for a given cell type. The methods of the present invention also advantageously allow one to assay or screen for desired cell types by characterizing and comparing the expression profiles from a plurality of cells and alternatively, comparing expression profiles obtained according to the present invention with a database of known expression profiles for a given cell type.

1. Sample Preparation

To measure the transcription level (and thereby the expression level) of a gene or genes, a nucleic acid sample comprising mRNA transcript(s) of the gene or genes, or nucleic acids derived from the mRNA transcript(s) is provided. A nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include mRNA transcripts of the gene or genes, cDNA reverse transcribed from the mRNA, cRNA transcribed from the cDNA, DNA amplified from the genes, RNA transcribed from amplified DNA, and the like. In some methods, a nucleic acid sample is the total mRNA isolated from a biological sample. The term “biological sample”, as used herein, refers to a sample obtained from an organism or from components (e.g., cells) or an organism. The sample can be of any biological tissue or fluid. Frequently the sample is from a patient. Such samples include sputum, blood, blood cells (e.g., white cells), tissue or fine needle biopsy samples, urine, peritoneal fluid, and fleural fluid, or cells therefrom. Biological samples can also include sections of tissues such as frozen sections taken for histological purposes. Often two samples are provided for purposes of comparison. The samples can be, for example, from different cell or tissue types, from different species, from different individuals in the same species or from the same original sample subjected to two different treatments (e.g., drug-treated and control).

2. Method

(a.) Generation of cDNAs

For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part 1. Theory and Nucleic Acid Preparation, P. Tijssen, ed. Elsevier, N.Y. (1993)).

The total nucleic acid can be isolated from a given sample using, for example, an acid quanidinium-phenol-choloroform extraction method and poly A⁺ mRNA is isolated by oligo dT column chromatography or by using (dT)ₙ magnetic beads (see, e.g., Sambrook et al., Molecular Cloning: A Laboratory Manual (2^{nd} ed.), Vols 1-3, Cold Spring Harbor Laboratory, (1989), or Current Protocols in Molecular Biology, F. Ausubel et al., ed., Breene Publishing and Wiley-Interscience, N.Y. (1987)).

The sample mRNA can be reverse transcribed with a reverse transcriptase and a primer consisting of oligo dT and a sequence encoding the phage T7 promoter to provide single stranded DNA template. The second DNA strand is polymerized using a DNA polymerase. Methods of in vitro polymerization are well known (see, e.g., Sambrook, supra) and this particular method is described in detail by Van Gelder, et al., Proc. Natl. Acad. Sci. U.S.A 87: 1663-1667 (1990) which report that in vitro amplification according to this method preserves the relative frequencies of the various RNA transcripts. Eberwine et al, Proc. Natl. Acad. Sci. U.S.A (1992) 89:3010-3014 provide a further protocol that uses two round of amplification via in vitro transcription thereby permitting expression monitoring. Eberwine et al. describe another method of amplification in Methods (1996) 10(3): 283-8. Another method of amplification is described in Dixon et al., Nucleic Acids Res (1998) 26(19): 4426-31. A still further method of amplification is the amplification method described in Dulac et al., Cell (1995) 83: 195-206. Alternative methods of amplification are described in U.S. Ser. No. 60/126,796 filed on Mar. 30, 1999; Brady, G. et al., Methods in Molecular and Cellular Biology 2:17-25 (1990); Brady, G. et al., Current Biology (1995) 5:909-922 which is herein incorporated by reference). In some methods, individual cells or single cell populations are obtained by tissue biopsies or microdissection. In other methods single cells are obtained by cell sorting. In other methods, single cells are obtained by serial dilution. Preferred methods of cDNA synthesis and amplification are described in Dulac, C. (Curr Top Dev Biol. (1998) 36:245-58 1998); this reference and all references cited therein are herein incorporated by reference). Nucleic acids are typically labeled. Label can be introduced during amplification either by linkage to one of the primers or by one of the nucleotides being incorporated. Alternatively, labeling can be effected after amplification and cleavage by end-labeling. Detectable labels suitable for use in the present invention include any composition detectable by spectroscopic, photochemical, biochemical, immunochemical, electrical, optical or chemical means; see WO 97/10365.

Preferred methods achieve amplification of the entire population of polyA⁺ RNA within a single cell being analyzed. The preferred methods also provide for linear amplification which preserves the species of mRNA being amplified.

The PCR method of amplification is described in PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes). Nucleic acids in a target sample are usually labeled in the course of amplification by inclusion of one or more labeled nucleotides in the amplification mix. Labels can also be attached to amplification products after amplification e.g., by end-labeling. The amplification product can be RNA or DNA depending on the enzyme and substrates used in the amplification reaction.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. U.S.A 86, 1173 (1989)), and self-sustained sequence replication (Guatelli et al., Proc. Nat. Acad. Sci. U.S.A 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

A variety of labels can be incorporated into target nucleic acids in the course of amplification or after amplification. Suitable labels include fluorescein or biotin, the latter being detected by staining with phycoerythrin-streptavidin after hybridization. In some methods, hybridization of target nucleic acids is compared with control nucleic acids. Optionally, such hybridizations can be performed simultaneously using different labels for target and control samples. Control and target samples can be diluted, if desired, prior to hybridization to equalize fluorescence intensities.

3. Supports

Supports can be made of a variety of materials, such as glass, silica, plastic, nylon or nitrocellulose. Supports may be nonporous or porous and may take the shape of films, rods, beads, threads, wires and other support shapes known to those skilled in the art. Supports are preferably rigid and have a planar surface. Supports typically have from 1-10,000,000 discrete spatially addressable regions, or synthesis cells. Supports having 10-1,000,000 or 100-100,000 or 1000-100,000 synthesis cells are common. The density of synthesis cells is typically at least 1000, 10,000, 100,000 or 1,000,000 synthesis cells within a square centimeter. Typically, a single type of probe is present per synthesis cell. In some supports, all synthesis cells are occupied by pooled mixtures of probes. In other supports, some synthesis cells are occupied by pooled mixtures of probes, and other synthesis cells are occupied, at least to the degree of purity obtainable by synthesis methods, by a single type of polynucleotide. The strategies for probe design described in the present application can be combined with other strategies, such as those described by WO 95/11995, EP 717,113 and WO 97/29212 in the same array.

The location and sequence of each different polynucleotide probe in the array is generally known. Moreover, the large number of different probes can occupy a relatively small area providing a high density array having a probe density of generally greater than about 60, more generally greater than about 100, and most generally greater than about 600, often greater than about 1000, more often greater than about 5,000, most often greater than about 10,000, preferably greater than about 40,000 more preferably greater than about 100,000, and most preferably greater than about 400,000 different polynucleotide probes per cm². The small surface area of the array (often less than about 10 cm², preferably less than about 5 cm² more preferably less than about 2 cm², and most preferably less than about 1.6 cm²) permits the use of small sample volumes and extremely uniform hybridization conditions.

4. Synthesis of Probe Arrays

Arrays of probes can be synthesized in a step-by-step manner on a support or can be attached in presynthesized form. Arrays of probes according to the present invention include miniaturized arrays or microarrays. A preferred method of synthesis is VLSIPS™ (see Fodor et al., 1991, Fodor et al., 1993, Nature 364, 555-556; McGall et al., U.S. Ser. No. 08/445,332; U.S. Pat. No. 5,143,854; EP 476,014), which entails the use of light to direct the synthesis of polynucleotide probes in high-density, miniaturized arrays. Algorithms for design of masks to reduce the number of synthesis cycles are described by Hubbel et al., U.S. Pat. No. 5,571,639 and U.S. Pat. No. 5,593,839. Arrays can also be synthesized in a combinatorial fashion by delivering monomers to cells of a support by mechanically constrained flowpaths. See Winkler et al., EP 624,059. Arrays can also be synthesized by spotting monomers reagents on to a support using an ink jet printer. See id.; Pease et al., EP 728,520.

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., Trulson et al., U.S. Pat. No. 5,578,832; Stern et al., U.S. Pat. No. 5,631,734 and are available from Affymetrix, Inc., under the GENECHIP label. Some types of label provide a signal that can be amplified by enzymatic methods (see Broude, et al., Proc. Natl. Acad. Sci. U.S.A. 91, 3072-3076 (1994))

5. Design of Arrays

(a.) Customized and Generic Arrays

The design of arrays for expression monitoring is generally described, for example, in e.g., WO 97/27317 and WO 97/10365. There are two principal categories of arrays. One type of array detects the presence and/or levels of particular mRNA sequences that are known in advance. In these arrays, polynucleotide probes can be selected to hybridize to particular preselected subsequences of mRNA gene sequence. Such expression monitoring arrays can include a plurality of probes for each mRNA to be detected. For analysis of mRNAs, the probes are designed to be complementary to the region of the mRNA that is contained in the target nucleic acids (i.e., the 3′ end or at a location a distance away from the 3′ end). The array can also include one or more control probes.

The other type of array is sometimes referred to as a generic array in the sense that the array can be used to analyze mRNAs irrespective of whether the sequence of an mRNA or mRNA tag is known in advance. Such arrays can include random, haphazardly selected, or arbitrary probe sets. Alternatively, a generic array can include all possible polynucleotides of a particular pre-selected length. A random polynucleotide array is an array in which the pool of nucleotide sequences of a particular length does not significantly deviate from a pool of nucleotide sequences selected in a random manner (i.e., blind, unbiased selection) from a collection of all possible sequences of that length. Arbitrary or haphazard nucleotide arrays of polynucleotide probes are arrays in which the polynucleotide probe selection is selected without identifying and/or preselecting target nucleic acids. Arbitrary or haphazard nucleotide arrays can approximate or even be random, however there in no assurance that they meet a statistical definition of randomness. The arrays can reflect some nucleotide selection based on probe composition, and/or non-redundancy of probes, and/or coding sequence bias as described herein. However such probe sets are still not chosen to be specific for any particular genes.

Alternatively, generic arrays can include all possible nucleotides of a given length; that is, polynucleotides having sequences corresponding to every permutation of a sequence. Thus since the polynucleotide probes of this invention preferably include up to 4 bases (A, G, C, T) or (A, G, C, U) or derivatives of these bases, an array having all possible nucleotides of length X contains substantially 4^{X} different nucleic acids (e.g., 16 different nucleic acids for a 2 mer, 64 different nucleic acids for a 3 mer, 65536 different nucleic acids for an 8 mer). Some small number of sequences can be absent from a pool of all possible nucleotides of a particular length due to synthesis problems, and inadvertent cleavage). An array comprising all possible nucleotides of length X refers to an array having substantially all possible nucleotides of length X. All possible nucleotides of length X includes more than 90%, typically more than 95%, preferably more than 98%, more preferably more than 99%, and most preferably more than 99.9% of the possible number of different nucleotides. Generic arrays are particularly useful for comparative hybridization analysis between two mRNA populations or nucleic acids derived therefrom.

(b) Variations

(1) Constant Regions

In both customized and generic array, probes can comprise additional constant regions fused with the variable regions that mediate hybridization to target nucleic acid. In some arrays, constant regions are double stranded thereby providing a site at which hybridized target can ligate to immobilized probes. A constant domain is a nucleotide subsequence that is common to substantially all of the polynucleotide probes. Constant domains are typically located at the terminus of the polynucleotide probe closest to the substrate (i.e., attached to the linker/anchor molecule). The constant regions can comprise virtually any sequence. Some constant regions comprise a sequence or subsequence complementary to the sense or antisense strand of a restriction site (a nucleic acid sequence recognized by a restriction enzyme).

Constant regions can be synthesized de novo on the array or prepared in a separate procedure and then coupled intact to the array. Since the constant domain can be synthesized separately and then the intact constant subsequences coupled to the high density array, the constant domain can be virtually any length. Some constant domains range from 3 nucleotides to about 500 nucleotides in length, more typically from about 3 nucleotides in length to about 100 nucleotides in length, most typically from 3 nucleotides in length to about 50 nucleotides in length. Constant domains can also range from 3 nucleotides to about 45 nucleotides in length, or from 3 nucleotides in length to about 25 nucleotides in length or from 3 to about 15 or even 10 nucleotides in length. Constant domains can also range from about 5 nucleotides to about 15 nucleotides in length.

(2) Control Probes

Either customized or generic probe arrays can contain control probes in addition to the probes described above.

(a.) Normalization Controls

Normalization controls are typically perfectly complementary to one or more labeled reference polynucleotides that are added to the nucleic acid sample. The signals obtained from the normalization controls after hybridization provide a control for variations in hybridization conditions, label intensity, reading and analyzing efficiency and other factors that can cause the signal of a perfect hybridization to vary between arrays. Signals (e.g., fluorescence intensity) read from all other probes in the array can be divided by the signal (e.g., fluorescence intensity) from the control probes thereby normalizing the measurements.

Virtually any probe can serve as a normalization control. However, hybridization efficiency can vary with base composition and probe length. Normalization probes can be selected to reflect the average length of the other probes present in the array, however, they can also be selected to cover a range of lengths. The normalization control(s) can also be selected to reflect the (average) base composition of the other probes in the array. However one or a fewer normalization probes can be used and they can be selected such that they hybridize well (i.e., no secondary structure) and do not match any target-specific probes.

Normalization probes can be localized at any position in the array or at multiple positions throughout the array to control for spatial variation in hybridization efficiently. The normalization controls can be located at the corners or edges of the array as well as in the middle of the array.

(b.) Expression Level Controls

Expression level controls can be probes that hybridize specifically with constitutively expressed genes in the biological sample. Expression level controls can be designed to control for the overall health and metabolic activity of a cell. Examination of the covariance of an expression level control with the expression level of the target nucleic acid can indicate whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse can also be true. Thus where the expression levels of both an expression level control and the target gene appear to both decrease or to both increase, the change can be attributed to changes in the metabolic activity of the cell as a whole, not to differential expression of the target gene in question. Conversely, where the expression levels of the target gene and the expression level control do not co-vary, the variation in the expression level of the target gene can be attributed to differences in regulation of that gene and not to overall variations in the metabolic activity of the cell.

Virtually any constitutively expressed gene can provide a suitable target for expression level controls. Typically expression level control probes can have sequences complementary to subsequences of constitutively expressed genes including, but not limited to the β-actin gene, the transferrin receptor gene, the GAPDH gene, and the like.

(c.) Mismatch Controls

Mismatch controls can also be provided for the probes to the target genes, for expression level controls or for normalization controls. Mismatch controls are typically employed in customized arrays containing probes matched to known mRNA species. For example, some such arrays contain a mismatch probe corresponding to each match probe. The mismatch probe is the same as its corresponding match probe except for at least one position of mismatch. A mismatched base is a base selected so that it is not complementary to the corresponding base in the target sequence to which the probe can otherwise specifically hybridize. One or more mismatches are selected such that under appropriate hybridization conditions (e.g. stringent conditions) the test or control probe can be expected to hybridize with its target sequence, but the mismatch probe cannot hybridize (or can hybridize to a significantly lesser extent). Mismatch probes can contain a central mismatch. Thus, for example, where a probe is a 20 mer, a corresponding mismatch probe can have the identical sequence except for a single base mismatch (e.g., substituting a G, a C or a T for an A) at any of positions 6 through 14 (the central mismatch).

In generic (e.g., random, arbitrary, or haphazard) arrays, since the target nucleic acid(s) are unknown, perfect match and mismatch probes cannot be a priori determined, designed, or selected. In this instance, the probes can be provided as pairs where each pair of probes differ in one or more preselected nucleotides. Thus, while it is not known a priori which of the probes in the pair is the perfect match, it is known that when one probe specifically hybridizes to a particular target sequence, the other probe of the pair can act as a mismatch control for that target sequence. The perfect match and mismatch probes need not be provided as pairs, but can be provided as larger collections (e.g., 3, 4, 5, or more) of probes that differ from each other in particular preselected nucleotides.

In both customized and generic arrays mismatch probes can provide a control for non-specific binding or cross-hybridization to a nucleic acid in the sample other than the target to which the probe is complementary. Mismatch probes thus can indicate whether a hybridization is specific or not. For example, if the complementary target is present, the synthesis cells containing perfect match probes can be consistently brighter than those containing mismatch probes. In addition, if all central mismatches are present, the mismatch probes can be used to detect a mutation. Finally, the difference in intensity between the perfect match and the mismatch probe (I(PM)-I(MM)) can provide a good measure of the concentration of the hybridized material.

(d.) Sample Preparation Amplification, and Quantitation Controls

Arrays can also include sample preparation/amplification control probes. These can be probes that are complementary to subsequences of control genes selected because they do not normally occur in the nucleic acids of the particular biological sample being assayed. Suitable sample preparation/amplification control probes can include, for example, probes to bacterial genes (e.g., Bio B) where the sample in question is a biological sample from a eukaryote.

The RNA sample can then be spiked with a known amount of the nucleic acid to which the sample preparation/amplification control probe is directed before processing. Quantification of the hybridization of the sample preparation/amplification control probe can then provide a measure of alteration in the abundance of the nucleic acids caused by processing steps (e.g., PCR, reverse transcription, or in vitro transcription).

Quantitation controls can be similar. Typically they can be combined with the sample nucleic acid(s) in known amounts prior to hybridization. They are useful to provide a quantitation reference and permit determination of a standard curve for quantifying hybridization amounts (concentrations).

6. Methods of Detection

In one method of detection, mRNA or nucleic acid derived therefrom, typically in denatured form, are applied to an array. The component strands of the nucleic acids hybridize to complementary probes, which are identified by detecting label. Optionally, the hybridization signal of matched probes can be compared with that of corresponding mismatched or other control probes. Binding of mismatched probe serves as a measure of background and can be subtracted from binding of matched probes. A significant difference in binding between a perfectly matched probes and a mismatched probes signifies that the nucleic acid to which the matched probes are complementary is present. Binding to the perfectly matched probes is typically at least 1.2, 1.5, 2, 5 or 10 or 20 times higher than binding to the mismatched probes.

In a variation of the above method, nucleic acids are not labeled but are detected by template-directed extension of a probe hybridized to a nucleic acid strand with the nucleic acid strand serving as a template. The probe is extended with a labeled nucleotide, and the position of the label indicates, which probes in the array have been extended. By performing multiple rounds of extension using different bases bearing different labels, it is possible to determine the identity of additional bases in the tag than are determined through complementarity with the probe to which the tag is hybridized. The use of target-dependent extension of probes is described by U.S. Pat. No. 5,547,839.

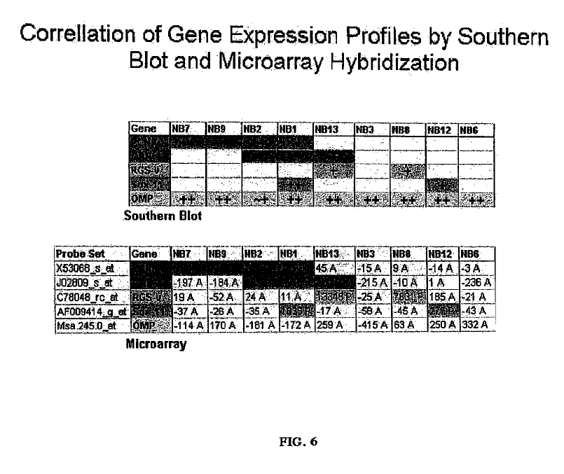

In a further variation, probes hybridized to tag strands are extended with inosine. Either the inosine or the tag strand can be labeled (see FIG. 6). The addition of degenerate bases, such as inosine (it can pair with all other bases), can increase duplex stability between the polynucleotide probe and the denatured single stranded DNA nucleic acids. The addition of 1-6 inosines onto the end of the probes can increase the signal intensity in both hybridization and ligation reactions on a generic ligation array. This can allow for ligations at higher temperatures. The use of degenerate bases is described in WO 97/27317.

Ligation reactions can offer improved discrimination between fully complementary hybrids and those that differ by one or more base pairs, particularly in cases where the mismatch is near the 5′ terminus of the polynucleotide probes. Use of a ligation reaction in signal detection increases the stability of the hybrid duplex, improves hybridization specificity (particularly for shorter polynucleotide probes (e.g., 5 to 12-mers), and optionally, provides additional sequence information. Ligation reactions used in signal detection are described in WO 97/27317. Optionally, ligation reactions can be used in conjunction with template-directed extension of probes, either by inosine or other bases.

7. Analysis of Hybridization Patterns

The position of label is detected for each probe in the array and accordingly the concentration of each sequence that is complementary to a probe on the array is determined by measuring the fluorescence intensity using a reader, such as described by U.S. Pat. No. 5,143,854, WO 90/15070, and Trulson et al., supra. For customized arrays, the hybridization pattern can then be analyzed to determine the presence and/or relative amounts or absolute amounts of known mRNA species in samples being analyzed as described in e.g., WO 97/10365. Comparison of the expression patterns of two samples is useful for identifying mRNAs and their corresponding genes that are differentially expressed between the two samples.

The quantitative monitoring of expression levels for large numbers of genes can prove valuable in elucidating gene function, exploring the causes and mechanisms of disease, and for the discovery of potential therapeutic and diagnostic targets. Expression monitoring can be used to monitor the expression (transcription) levels of nucleic acids whose expression is altered in a disease state. For example, a cancer can be characterized by the overexpression of a particular marker such as the HER2 (c-erbB-2/neu) protooncogene in the case of breast cancer.

Expression monitoring can be used to monitor expression of various genes in response to defined stimuli, such as a drug. This is especially useful in drug research if the end point description is a complex one, not simply asking if one particular gene is overexpressed or underexpressed. Therefore, where a disease state or the mode of action of a drug is not well characterized, the expression monitoring can allow rapid determination of the particularly relevant genes.

In generic arrays, the hybridization pattern is also a measure of the presence and abundance of relative mRNAs in a sample, although it is not immediately known, which probes correspond to which mRNAs in the sample.

However the lack of knowledge regarding the particular genes does not prevent identification of useful therapeutics. For example, if the hybridization pattern on a particular generic array for a healthy cell is known and significantly different from the pattern for a diseased cell, then libraries of compounds can be screened for those that cause the pattern for a diseased cell to become like that for the healthy cell. This provides a detailed measure of the cellular response to a drug.

Generic arrays can also provide a powerful tool for gene discovery and for elucidating mechanisms underlying complex cellular responses to various stimuli. For example, generic arrays can be used for expression fingerprinting. Suppose it is found that the mRNA from a certain cell type displays a distinct overall hybridization pattern that is different under different conditions (e.g., when harboring mutations in particular genes, in a disease state). Then this pattern of expression (an expression fingerprint), if reproducible and clearly differentiable in the different cases can be used as a very detailed diagnostic. It is not required that the pattern be fully interpretable, but just that it is specific for a particular cell state (and preferably of diagnostic and/or prognostic relevance).

Both customized and generic arrays can be used in drug safety studies.

For example, if one is making a new antibiotic, then it should not significantly affect the expression profile for mammalian cells. The hybridization pattern can be used as a detailed measure of the effect of a drug on cells, for example, as a toxicological screen. The sequence information provided by the hybridization pattern of a generic array can be used to identify genes encoding mRNAs hybridized to an array. Such methods can be performed using DNA nucleic acids of the invention as the target nucleic acids described in WO 97/27317. DNA nucleic acids can be denatured and then hybridized to the complementary regions of the probes, using standard conditions described in WO 97/27317. The hybridization pattern indicates which probes are complementary to nucleic acid strands in the sample. Comparison of the hybridization pattern of two samples indicates which probes hybridize to nucleic acid strands that derive from mRNAs that are differentially expressed between the two samples. These probes are of particular interest, because they contain complementary sequence to mRNA species subject to differential expression. The sequence of such probes is known and can be compared with sequences in databases to determine the identity of the full-length mRNAs subject to differential expression provided that such mRNAs have previously been sequenced. Alternatively, the sequences of probes can be used to design hybridization probes or primers for cloning the differentially expressed mRNAs. The differentially expressed mRNAs are typically cloned from the sample in which the mRNA of interest was expressed at the highest level. In some methods, database comparisons or cloning is facilitated by provision of additional sequence information beyond that inferable from probe sequence by template dependent extension as described above.

8. Kits

The invention further provides kits comprising probe arrays as described above. Optional additional components of the kit include, for example, other restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

### EXAMPLE I

### cDNA Synthesis From Single Neurons

The following general protocol known to those skilled in the art was used to perform a differential screen of cDNA libraries prepared from single olfactory neurons isolated from the rat vomeronasal organ. See Dulac, C. and Axel, R., Cell, 83, 195-206, 1995.

Target neuroepithelium tissue was microdissected and gently dissociated using a very mild trypsin solution to obtain a single cell suspension in which neuron still bear their axon and dendrites and can therefore be selected on an individual basis based on their morphology.

An individual cell was then selected and added to lysis buffer which resulted in lysing of the cell. Cell RNA was then primed with an oligodT primer.

Reverse transcription with reverse transcriptase was then performed in limiting conditions of time and reagents to facilitate incomplete extension and to prepare short cDNA of between about 500 bp to about 1000 bp and more particularly, about 600 bp. Incomplete extension can be obtained by using short extension times insufficient to make complete extension. For example, an extension time of 10 seconds can be used for a typical population of mRNA. Alternatively, incomplete extension can be achieved by using a suboptimal temperature for the polymerase effecting extension. In a further variation, incomplete extension can be achieved by using terminator nucleotides such as dideoxynucleotides. The conditions of incomplete extension typically result in extended nucleic acids having lengths between about 100 to about 1000 bases. In some methods, incomplete extension typically results in extended nucleic acids having lengths between about 400 to about 800 bases. In some methods, incomplete extension results in extended nucleic acids having lengths about 600 bases. The cDNA was then tailed at the 5′ end with multiple dATP using polyA (dATP) and terminal transferase.

The cDNA was then amplified with PCR reagents using a 60mer primer having 24(dT) at the 3′ end. PCR cycling was performed at 94° C. for 1 minute, then 42° C. for 2 minutes and then 72° C. for 6 minutes with 10 second extension times at each cycle. 25 cycles were performed. The additional Taq polymerase was added and an additional 25 cycles were performed.

The method disclosed in Dulac, Current Topics in Developmental Biology, Cloning of Genes from Single Neurons 36:245-258 (1998) is instructive in the preparation of the cDNA of the present invention. According to the present invention, the neuroepithelium was dissected under the microscope, placed in a 35-mm petri dish, and rinsed several times in phosphate-buffered saline (PBS) without Ca⁺² and Mg⁺². The tissue was then fragmented into many small fragments with fine forceps, microscalpels, or microscissors. The PBS was then removed with a “pipetman” or a Pasteur pipette and replaced by 2 ml of PBS without Ca⁺² and Mg⁺² containing 0.025% trypsin, 0.75 mM ethylenediamine tetraacetic acid (Low Trypsin-High EDTA solution from Specialty Media) prewarmed at 37° C. Tissue and tryspin were mixed very gently by pipetting up and down two or three times with a 2-ml plastic pipette.

The petri dish containing the dissociating tissue was kept in a 37° C. incubator for 10 to 15 minutes. After 15 minutes, the tissue and trypsin were again mixed with a pipette very gently two or three times as before and the observed under an inverted microscope to reveal large clumps of cells. The dissociation was stopped when cells at the periphery of the large clumps were observed to start to dissociate and some fully dissociated cells were observed at the bottom of the petri dish. At this stage, if the clumps of cells are still very cohesive after 20 to 30 minutes, then remove the tryspin with a pipette, again add 2 ml of prewarmed trypsin, and keep 10 more minutes at 37° C.

To stop the trypsinization, the 2 ml of tryspin and tissue were transfered with a pipette into a 10-ml solution of prewarmed Dulbecco's modified Eagle's medium +10% fetal calf serum. Trituration was not performed at this stage. Instead, the trypsin and tissue were centrifuged for 10 minutes at 2000 rpm, all supernatant was removed, and 5 ml of cold PBS without Ca⁺² and Mg⁺² was added. The cell suspension was then triturated very gently by pipetting up and down four or five times with pipettes and pipetman tips of gradually smaller diameters: 2-ml plastic pipette, 1-ml plastic pipette, then 1 ml followed by a 200-μl-tip pipetman. The cell suspension was then kept on ice.

The cells were then observed on a Leitz inverted microscope to reveal clumps and isolated neurons retaining intact axonal and dendritic processes. The cell suspension was decanted for 10 minutes to remove the clumps of cells.

An appropriate dilution of the cell suspension was observed on a Leitz inverted microscope and neurons were identified by their round cell body and long axonal and dendritic processes. Isolated neurons were picked with a Leitz micromanipulator fitted with a pulled and beveled microcapillary, or directly with a mouth pipette connected to a pulled 25-μl microcapillary. Cells have to be quite sparse; otherwise, additional cells are likely to be picked at the same time or stick to the outside of the pipette. Successful picking of individual cells require only a few hours training.

In picking the cells, a four-well Multidish (Nunc) with 500 μl of PBS in each were used, so the focus of the microscope does not have to be changed from one well to the other. The candidate neuron was transferred from the well containing the cell suspension to the adjacent well containing no cell. The microcapillary was rinsed several times in a dish containing PBS, the cell was repicked and then seeded in a PCR tube.

Single cells or groups of 10 to 20 cells were seeded in a volume of 0.2 to 0.5 μl into thin-walled PCR reaction tubes containing 4 μl of ice-cold lysis buffer prepared as described below. The PCR tubes are transparent enough so the tip of the micro capillary can be seen reaching the solution. The tubes were spun immediately for 30 seconds to make sure the cell contacted the lysis buffer and preferably was located at the bottom of the tube and did not stick to the tube wall. The PCR tubes including the collected cells were then kept on ice. A zero control tube with no cell in it was also prepared. It is also useful to prepare a few tubes with clumps of 10 to 20 cells as positive controls. Seeding of PCR tubes with cells should not exceed a few hours.

During cell dissociation, the cDNA lysis buffer was prepared as follows. For 100 μl of cDNA lysis buffer, the following were mixed together on ice: 20 μl of Moloney muzine leukemia virus (MMLV) reverse transcriptase+buffer 5× (Gibco-BRL), 76 μl of H₂O(RNAse, DNAse free, Specialty Media), 0.5 μl of Nonidet P40 (USB), 1 μl of PrimeRNase inhibitor (3‘5’ Incorporated), 1 μl of RNAguard (Pharmacia), and 2 μl of freshly made, 1/24 dilution of stock primer mix. The stock primer mix, kept aliquoted at −20° C., included 10 μl each of 100 mM dATP, dCTP, dGTP, dTTP solutions (12.5 mM final)(Boehringer); 10 μl of 50 OD/ml pd(T)19-24 (Pharmacia); and 30 μl H2O.

### cDNA Synthesis And Amplification

In general, individual neurons are picked with a microcapillary and directly seeded in PCR reaction tubes containing cell lysis buffer. Lysis is subsequently performed at 65° C., and oligodT-primed first-strand cDNA synthesis is achieved with the addition of a mixture of reverse transcriptases at 37° C., followed by reagents allowing the synthesis of a poly (A) tail in 5′ of the first-strand cDNA. The 5′ poly(A) and 3′ poly(T) tails allow PCR amplification to be performed using a primer containing a poly(T) sequence. This protocol, modified from Brady et al. (1990), allows more than 50 μg of PCR-amplified cDNA to be synthesized from individual neurons in a single tube (Dulac and Axel, 1995). The reverse transcription is performed in limiting conditions to generate cDNA of between about 500 bp and about 1 kb, which are then likely to be equally amplified. In this manner, and despite the PCR step, the amplified cDNA maintains an accurate representation of the different cell RNAs. This cDNA synthesis can be done on single cells or groups of cells, as well as on very small amounts of RNA purified from several hundred cells.

Specifically, the single cells collected in the PCR tubes were lysed at 65° C. for one minute, then the tubes were maintained for 1 to 2 minutes at room temperature to allow the oligodT primer to anneal to the RNA. The PCR tubes were then put back on ice and spun quickly at 4° C. for 2 minutes to remove the condensation. 0.5 μl of a 1:1 (vol:vol) mix of Avian myelo blastosis virus (AMV) reverse transcriptase (Gibco-BRL) and MMLV-reverse transcriptase were then added and incubated for a maximum of 15 minutes at 37° C. The enzymes were then inactivated for 10 minutes at 65° C., put back on ice, and spun 2 minutes at 4° C.

On ice, 4.5 μl of 2× tailing buffer containing 800 μl of 5× BRL terminal transferase buffer (Gibco-BRL), 30 μl of 100 mM dATP, and 1.17 ml H₂O was then added. The tubes were then incubated at 37° C. for 15 minutes. The enzymes were then inactivated for 10 minutes at 65° C., put back on ice, and spun 2 minutes at 4° C.

To each tube, 90 μl of ice-cold PCR buffer mix was added. It is important to keep all reagents and PCR buffer mix on ice to avoid primer dimer formation. The 90 μl of PCR buffer mix contained 10 μl of 10× PCR buffer II (Perkin Elmer), 10 μl of 25 mM MgCl₂ (Perkin Elmer) 0.5 μl of 20 mg/ml BSA (Boehriner), 1 μl of each 100 mM deoxynucleotide triphosphate (Boehringer), 1 μl of 5% Triton X 100(Sigma), 5 μg of AL1 primer (ATT GGA TCC AGG CCG CTC TGG ACA AAA TAT GAA TTC (T)₂₄ (0.1M scale)(Oligo etc.), H₂O qsp 90 μl, 2 μl of AmpliTaq (Perkin-Elmer), and 1 or 2 drops of mineral oil, molecular biology grade (Sigma).

On a Perkin Elmer DNA Thermal Cycler 480, 25 cycles were performed as follows: 94° C. for 1 minute, 42° C. for 2 minutes, 72° C. for 6 minutes, with a 10-second extension time at each cycle. When these 25 first cycles were finished, 1 μl of AmpliTaq was added directly to each tube and 25 more cycles were performed with the same program as before but without the extension time at each cycle. Higher yields are generally obtainable when the second set of cycles is started as soon as the first set of cycles is completed.

cDNA was extracted in phenol-chloroform, precipitated with ethanol and then half of the sample was frozen at −80° C. as a stock to avoid thawing and freezing the entire amount of cDNA while analyzing it.

### Differential Screening of Single Cell Libraries

To check the quality of the cDNA obtained, two agarose gels 1.5% with 5 μl of cell cDNA in each well were run. A very intense smear of DNA (around 500 ng) was observed from 0.4 to 1.2 kb. It is not unusual to find a similar result with the zero control which may result from some minor bacterial contaminants present in the enzyme solutions, but no specific probe should hybridize to that lane in further controls. The cDNA was then transferred to 4 Hybond N+ membranes (Amersham) in two double-sandwich Southern blots and hybridized with highly expressed ubiquitous genes (e.g., tubulin and a riboprotein), an ubiquitous gene expressed at a moderate or lower level (i.e., Go), and two genes specific of the cell type of interest. Since the cDNAs generated were mostly shorter than 1 kb, the probes or the PCR primers should correspond to the 3′ end of the genes tested. In addition, cross-hybridizations between different animal species at the 3′ untranslated region are unlikely, even between rat and mouse, even for very conserved genes like tubulin.

Signals for tubulin and riboprotein were extremely intense and appeared after less than 1 hour of exposure. Dividing cells can reduce levels of these markers. Additional markers can be used and will be apparent to one skilled in the art.

### Reamplification of Single Cell cDNA Samples

Each cell cDNA was reamplified according to the following protocol. Each single cell cDNA sample underwent three 100 ul PCR reactions. For one reaction the following PCR mix was combined: 80 ul of ultrapure H₂O, 10 ul of 10× PCR buffer, 10 ul of 10× MgCl₂, 0.2 ul each of dNTP, 1 ul of Tap polymerase and 5 ug of AL-1 primer.

100 ul of the mix was added to a negative control containing no DNA. 300 ul of the mix was added to a PCR tube for each single cell cDNA sample. 2.25 ul of stock single cell cDNA sample was then added to each 300 ul sample of mix and then divided into three 100 ul aliquots. 2 drops of mineral oil was then added to each 100 ul aliquot which were then amplified for 25 cycles at 94° C. for 1.5 minutes, 42° C. for 2 minute, 72° C. for 3 minutes, 72° C. for 20 minutes. The resulting product was then maintained at 4° C. Each PCR reaction product was then purified using a Quiaquick PCR purificiation kit from Quiagen.

### EXAMPLE II

### Gene Expression Profiling Using GENECHIP Expression Arrays

The following general protocol including the steps of fragmentation, end-labeling, hybridization and expression profiling was used to obtain an expression profile on a GENECHIP expression array.

5 μg (18 μl) of a PCR product (MOE-10 (0.275 μg/μl)) was combined with 15.5 μl EF sln (Tris in Qiagen kit PCR purification), 4 μl of 10× One-Phor-All buffer from Promega, and 0.5 units of DNase I. The total volume was 40 μl and included 5 μg of DNA and 0.50 μg of DNase I.

The total volume was then held at 37° C. for 14 minutes, then held at 99° C. for 15 minutes and then put on ice for 5 minutes to fragment the PCR product into segments about 50 bp to about 100 bp in length. The fragments were then end-labeled by combining the total volume with 1 μl of Biotin-N₆-ddATP (“NEN”) and 1.5 μl of TdT (terminal transferase) (15 unit/ul). The total volume (42.5 μl) was then held at 37° C. for 1 hour, then held at 99° C. for 15 minutes and then held on ice for 5 minutes.

The labeled and fragmented cDNA was hybridized with additional chips in 200 microliter of hybridization solution containing 5-10 microgram labeled target in 1× MES buffer (0.1 M MES, 1.0 M NaCl, 0.01% Triton X-100, pH 6.7) and 0.1 mg/ml herring sperm DNA. The arrays used were Affymetrix mouse expression arrays: 11K set (11KsubA and 11KsubB) which contain aproximately 11,000 genes and ESTs. Arrays were placed on a rotisserie and rotated at 60 rpm for 16 hours at 45° C. Following hybridization, the arrays were washed with 6× SSPE-T (0.9 M NaCl, 60 mM NaH2PO4, 6 mM EDTA, 0.005% Triton X-100, pH 7.6) at 22° C. on a fluidics station (Affymetrix) for 10×2 cycles, and then washed with 0.1 MES at 45° C. for 30 min. The arrays were then stained with a streptavidin-phycoerythrin conjugate (Molecular Probes), followed by 6× SSPE-T wash on the fluidics station for 10×2 cycles again. To enhance the signals, the arrays were further stained with Anti-streptavidin antibody for 30 min followed by a 15 min staining with a streptavidin-phycoerythrin conjugate again. After 6× SSPE-T wash on the fluidics station for 10×2 cycles, the arrays were scanned at a resolution of 3 μm using a modified confocal scanner (Affymetrix).

Additional PCR products were also subjected to fragmentation, end-labeling, hybridization and expression profiling as described above. The table below summarizes the results obtained for each single cell PCR product.

Array Type: Mu11KB

| | | Percentage of Genes |
|---|---|---|
| Cell Type | File Name | Expressed (P %) |
| NB 8 (newborn MOE cell) | YC031521 | 11.2% |
| SC 16 (VNO neuron) | YC031531 | 8.5% |
| SC 26 (VNO neuron) | YC031541 | 9.5% |
| Photoreceptor Cell | YC031551 | 5.5% |

Array Type: Mu11KA

| | | Percentage of Genes |
|---|---|---|
| PCR Product | Sample # | Expressed (P %) |
| NB 8 (newborn MOE cell) | YC031721 | 18.4% |
| SC 16 (VNO neuron) | YC031731 | 17.0% |
| SC 26 (VNO neuron) | YC031741 | 16.9% |
| Photoreceptor Cell | YC031551 | 6.4% |

Additional experiments were conducted to determine the number of expressed genes for single neurons and olfactory epithelium using the methods described above and the data is presented in Table II below. The olfactory epithelium tissue was not subjected to the amplification step described above.

| Experiment | # Expressed Genes | P % |
|---|---|---|
| Olfactory Epithelium | 3602 | 27.7% |
| Single Olfactory Neuron (NB1) | 2337 | 17.9% |
| Single Olfactory Neuron (I-11) | 1986 | 15.2% |
| Single VNO Neuron (Sc-16) | 1617 | 12.4% |

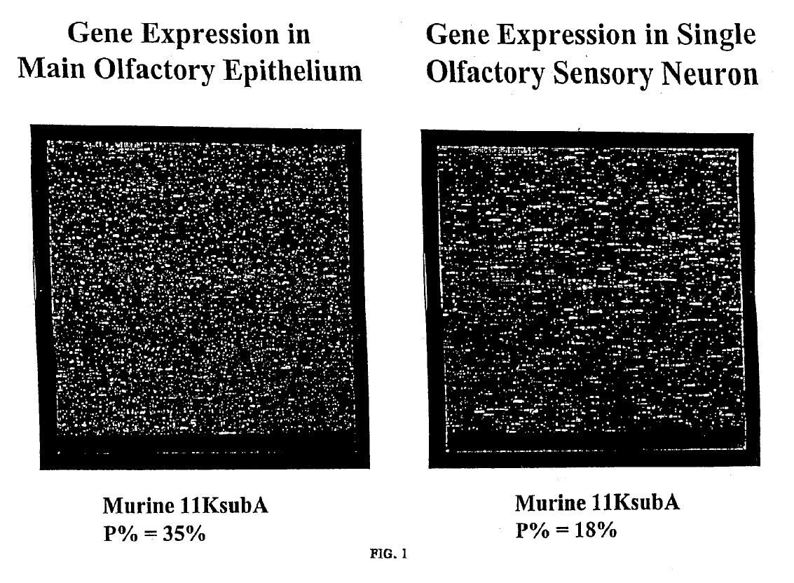

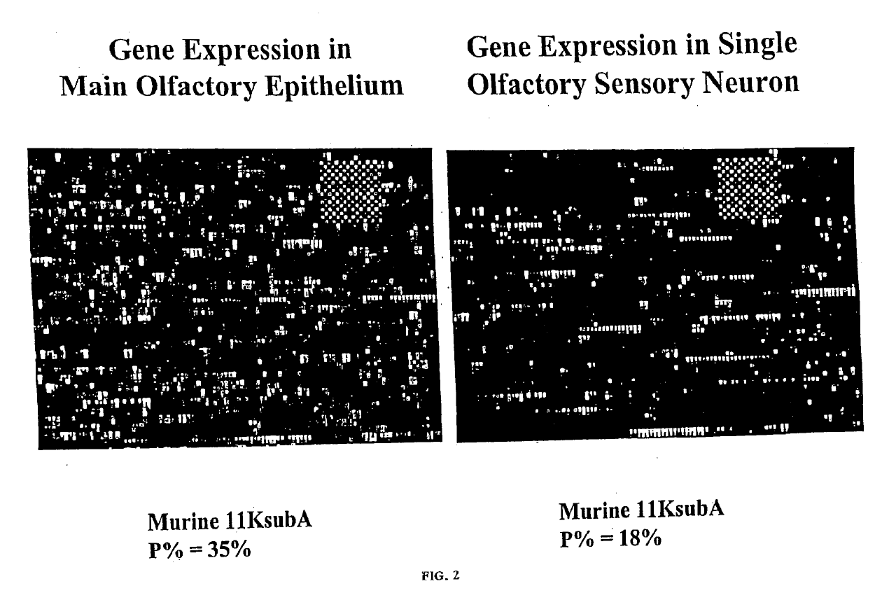

FIG. 1 shows a comparison of gene expression images of main olfactory epithelium and single olfactory sensory neuron. Identical murine 11K subA arrays were used to assess the gene expression of approximately 6,500 genes in both main olfactory epithelium (MOE) and single olfactory sensory neuron. 10 μg of labeled RNA target prepared from MOE was used for the left panel hybridization and 35% of the genes on the array were detected. 10 μg of labeled DNA target prepared from a single neuron is hybridized to the array on the right panel and 18% of the genes were detected. FIG. 2 shows identical regions of the arrays of FIG. 1. The hybridization results for the single neuron are significantly less complex and correspondingly more specific for the single neuron as compared with the main olfactory epithelium.

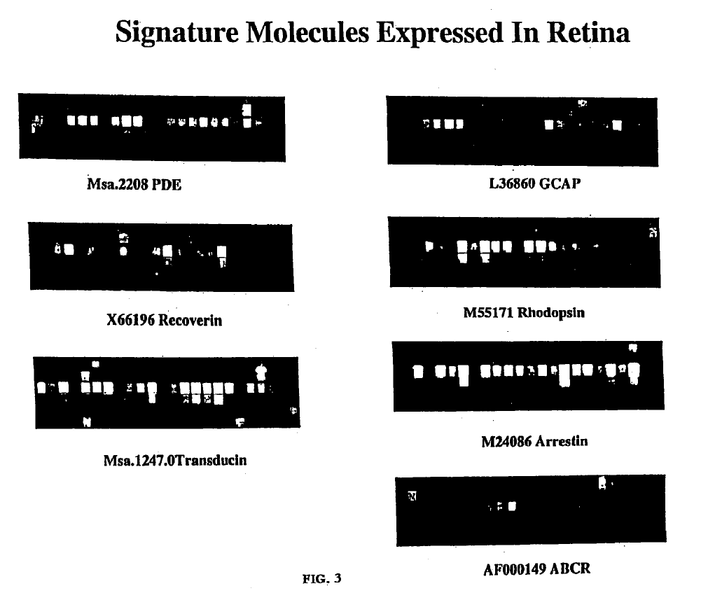

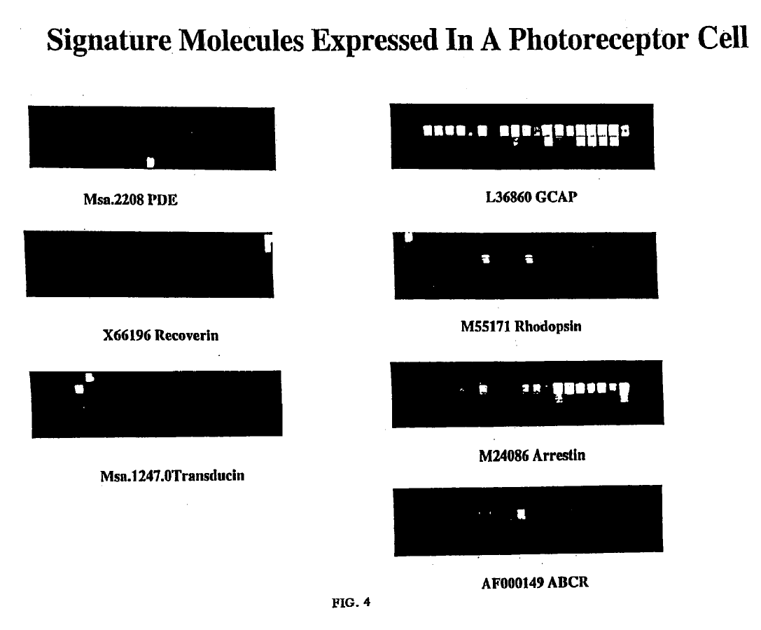

Images of several signature molecules expressed in the retina and in a single photoreceptor cell on murine arrays were obtained using the methods described above. The images are presented in FIGS. 3 and 4 which show less complexity in the images obtained for the photoreceptor cell.

### EXAMPLE III

### Confirmation of Linear Amplification of Single Cell cDNA

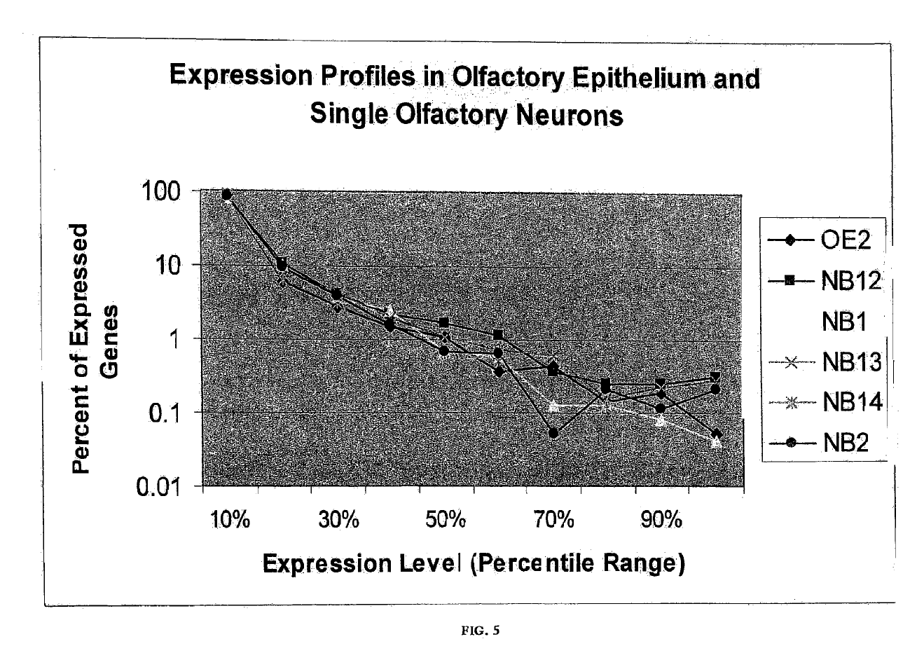

A number of experiments according to methods well known in the art were conducted to determine whether amplification of mRNA by methods described herein was linear. Gene expression profiles in olfactory epithelium and single olfactory neurons were compared. The data is presented in FIG. 5 and shows good correlation between the expression profiles of olfactory epithelium (OE2) and several single olfactory neurons (NB12, NB1, NB13, NB14, and NB2) in terms of percent of expressed genes versus expression level (percentile range).

### Correlation of Gene Expression Profiles by Southern Blot and Microarray Hybridization

Studies were conducted comparing the results of expression profiles of certain genes determined by Southern Blot methods with the results of expression profiles of the same genes determined by microarray hybridization according to the method of the present invention. The results are shown in FIG. 6 which indicates good correlation between the gene expression profiles obtained by Southern Blot and microarray hybridization confirming the utility of microarray hybridization methods for determining gene expression profiles for cells of interest.

### EXAMPLE VI

### Correlation Coefficient Analysis

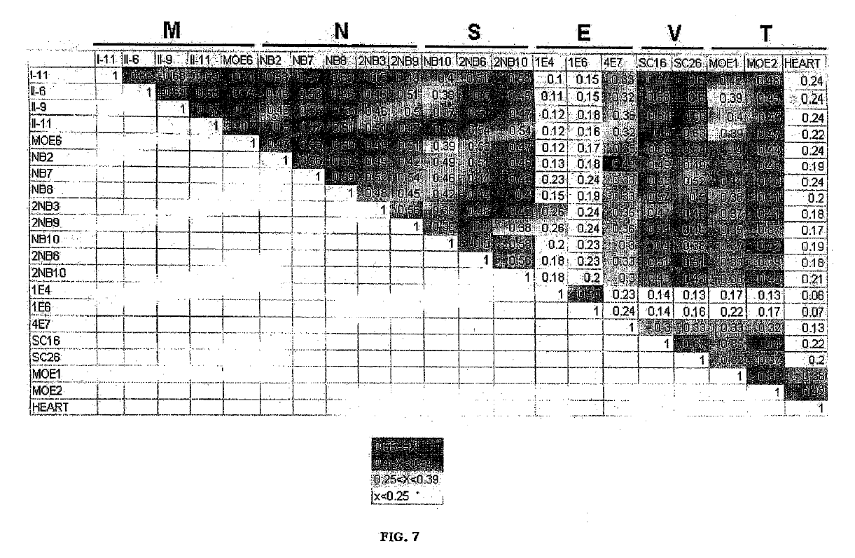

Single cells were picked from olfactory epithelium or vomeronasal epithelium, and single cell cDNA was prepared from each cell and hybridized to microarrays as described above. For tissues, whole RNA was reversed transcribed and hybridized to microarrays as described above. The change in expression profile for every gene among all cells was then measured, and the coefficient of correlation was obtained. The results are depicted in FIG. 7. M=olfactory sensory neurons (OSNs) picked from adult olfactory epithelium. N═OSNs picked from neonatal olfactory epithelium. S=supporting glial cells from olfactory epithelium. E=OSN progenitor cells. V=vomeronasal sensory neurons picked from adult vomeronasal epithelium. T=whole tissues. Higher correlation coefficients indicate single cell cDNA samples with more similar expression profiles. Cells which are expected to be more highly related tend to have higher correlation coefficients. For example, OSNs picked from adult olfactory epithelium tend to be highly correlated to each other but not to OSN progenitor cells. Alternatively, OSN progenitor cells tend to have low correlation to other sensory neurons and supporting cells but correlate very highly to each other.

### Hierarchical Clustering of Cells By Similarity of Expression Profile

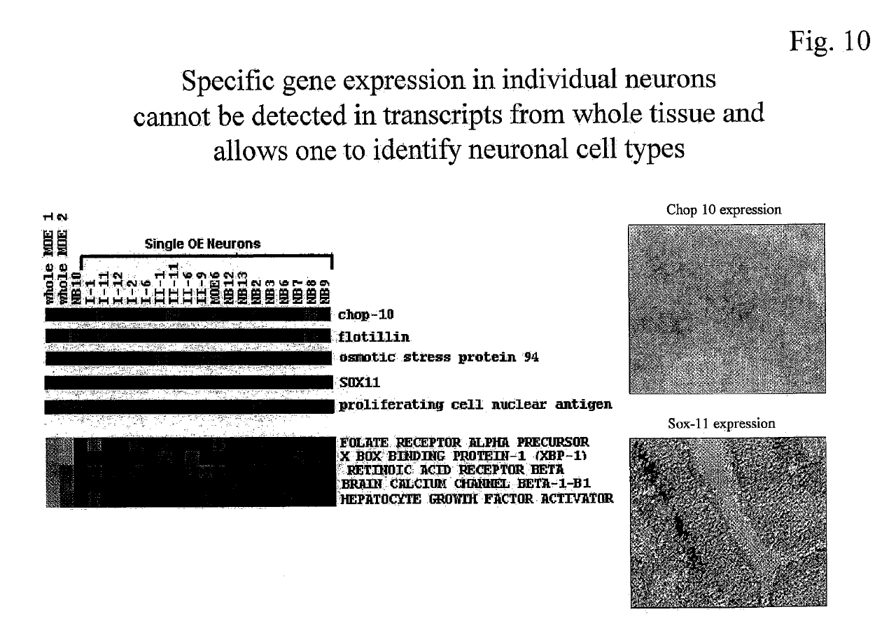

Using the correlation coefficient obtained, the relationship of individual cells, i.e. olfactory neurons from the main olfactory epithelium (MOE cells), two olfactory neurons from the vomeronasal organ (VNO cells), and one photoreceptor cell, was visualized by a hierarchical clustering analysis. The clustering is represented in FIG. 10, which shows that cells obtained from an embryonic MOE (I.) not only cluster together but also are different than cells obtained from a newborn MOE (II.), an adult MOE (IV.), and a photoreceptor cell. Additionally, the VNO cells (III) cluster together. NB10 is a supporting cell and therefore does not cluster with the other MOE cells. Gene Cluster and Tree View software available on-line from Stanford was used in the clustering analysis. Also, GeneCluster 1.0 software provided by the Whitehead/MIT Center for Genome Research can be used in clustering analysis.

### EXAMPLE VII

### Identification of the Nature and Function of a Cell by Monitoring its Transcriptional Profile

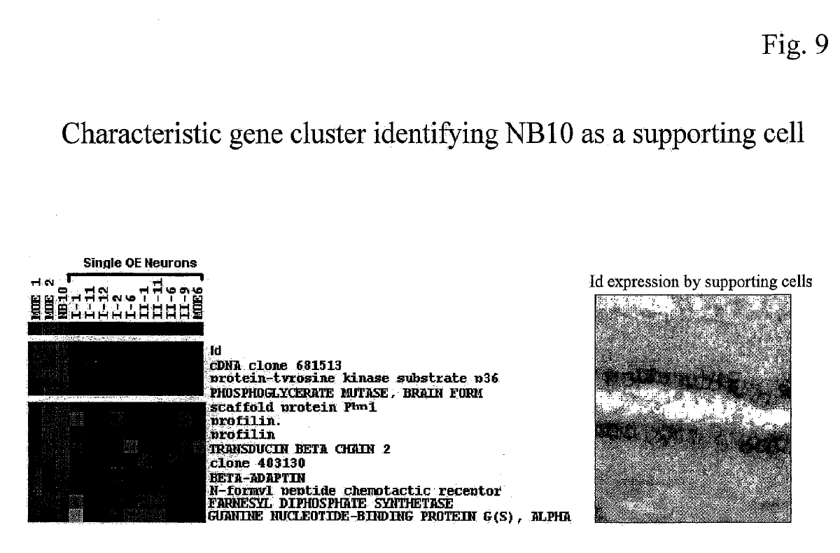

The monitoring of transcriptional profile from individual neurons, neuronal precursors and embryonic cells was carried out according to the methods described above. The nature and function of the cells were determined by comparing the expression profiles. FIG. 9 shows the expression of a set of genes by NB 10 and not by single olfactory neurons using hierarchical clustering software from Eisen et al. Previously incorporated by reference identifying NB 10 as a supporting cell. The expression pattern of Id by supporting cells of the olfactory epithelium is documented in the right panel. Using similar gene clustering methods, expression profiles of specific neuronal populations are identified in FIG. 12 and represent functionally or developmentally distinct neuronal subpopulations. These transcriptional signatures could not be identified from a large population of cells, such as whole olfactory MOE1 and MOE2.

Although the foregoing invention has been described in detail for purposes of clarity of understanding, it will be obvious that certain modifications can be practiced within the scope of the appended claims. All publications and patent documents cited above are hereby incorporated by reference in their entirety for all purposes to the same extent as if each were so individually denoted.

What is claimed is:

1. A method of monitoring expression of one or more genes in one or more cells, comprising:
contacting an array of probes with a population of nucleic acids derived from fewer than 1000 cells,
determining relative hybridization of the probes to the population of nucleic acids.

2. The method of claim 1, wherein the population of nucleic acids are derived from a single cell.

3. The method of claim 1, wherein the population of nucleic acids are prepared by reverse transcription of a population of mRNA to produce a population of cDNA and amplification of the cDNA.

4. The method of claim 3, wherein the reverse transcription is conducted under conditions of incomplete extension.

5. The method of claim 1, wherein the nucleic acids are derived from fewer than 100 cells.

6. The method of claim 4, wherein the conditions of incomplete extension are effected by use of limited reagents, by use of a shorter time than required for complete extension, by use of a suboptimal temperature, or by incorporation of a chain-terminating nucleotide.

7. The method of claim 4, wherein the conditions of incomplete extension synthesize polynucleotides with a median length of about 100-1000 bases.

8. The method of claim 4, wherein the conditions of incomplete extension synthesize polynucleotides with a median length of about 500-700 bases.

9. The method of claim 1, wherein the genes to be monitored are of at least partly known sequence, and the probe array comprises a probe set for each gene to be monitored, the probe set comprising a plurality of probes perfectly complementary to or perfectly matched to a transcript.

10. The method of claim 9, wherein at least some of the probes in each probe set are perfectly complementary to or perfectly matched to a segment within 1000 bases from the 3′ end of the sequence of the transcript.

11. The method of claim 9, wherein at least one probe in each probe set is perfectly complementary to or perfectly matched to a segment within 1000 bases from the 3′ end of the coding sequence of the transcript.

12. The method of claim 9, wherein the probes in the probe set are perfectly complementary to or perfectly matched to one gene, one gene family or one gene cluster in the plurality of transcripts to be monitored.

13. The method of claim 9, wherein at least ten probes in each probe set are perfectly complementary to or perfectly matched to a segment within 500 bases from the 3′ end of the coding sequence of the transcript.

14. The method of claim 9, wherein the probe array comprises a probe set for at least 1000 genes.

15. The method of claim 9, wherein the probe array comprises a probe set for at least 10,000 genes.

16. The method of claim 9, wherein each probe set further comprises a mismatch probe for each perfectly matched probe, the mismatched probe differing from the perfectly matched probe at a single position.

17. The method of claim 9, further comprising comparing the hybridization of matched and mismatched probes to determine the relative expression levels of the genes.

18. The method of claim 9, wherein the relative expression levels of each of 1000 genes are determined.

19. The method of claim 9, wherein the relative expression levels of each of 10,000 genes are determined.

20. The method of claim 9, wherein the relative expression levels of detected genes vary by at least about to 2-fold to about ten-fold.
